(19) United States
(12) Patent Application Publication
(10) Pub. No.: US 2018/0098991 A1
(43) Pub. Date: Apr. 12, 2018

(54) COMPOSITION FOR LIPID DISORDER TREATMENT

(71) Applicant: Bioenergy Healthcare GmbH, Koeln (DE)

(72) Inventors: Jochen HERGERT, Koeln (DE); Reinhold ROMBACH, Koeln (DE)

(73) Assignee: Bioenergy Healthcare GmbH, Koeln (DE)

(21) Appl. No.: 15/481,861

(22) Filed: Apr. 7, 2017

(30) Foreign Application Priority Data
Oct. 6, 2016 (AT) GM 50202/2016

(51) Int. Cl. A61K 31/519; A61K 36/28; A61K 31/435; A61K 31/05

(52) U.S. Cl. CPC A61K 31/519; A61K 36/28; A61K 2300/00; A61K 31/05; A61K 31/435

(57) ABSTRACT

A pharmaceutical composition for controlling the lipid metabolism or for the treatment or prevention of dislipidemia, dislipoproteinemia, or hypertriglyceridemia comprises folic acid and vitamin B₁₂ or physiologically acceptable derivatives or salts thereof, respectively.

### CROSS-REFERENCES TO RELATED APPLICATIONS

The present application claims priority of Patent Application No. GM 50202/2016, filed Oct. 6, 2016 in Austria, the entire contents of which are incorporated by reference herein.

### TECHNICAL FIELD

The present invention relates to a pharmaceutical composition for the treatment of lipid disorder, and in particular for the treatment or prevention of dislipidemia, dislipoproteinemia or hypertriglyceridemia, the composition containing folic acid, vitamin B12 and optionally vitamin B6, as well as to the use of a corresponding agent combination for manufacturing a medicament, a balanced diet or nutritional supplements.

### BACKGROUND ART

It is known, e. g. from document WO 98/50028 A1, to regulate plasma lipid levels considerably above normal values with the administration of fat soluble vitamins (vitamins A, D, E, K) and MTP-inhibitors. However, the efficacy, safety, and sustainability of this treatment is questionable.

### SUMMARY OF THE INVENTION

In order to overcome the above drawbacks of the prior art, the present invention instead suggests administering a composition of folic acid, vitamin B12 and optionally vitamin B6 in relatively high dosages (namely high in comparison to general guide values). An MTP-inhibitor is not required. Without wishing to be bound by theory, it is assumed that the effect is based on the following mechanisms: Vitamin B12 is required for transferring a 1-carbon-unit from folic acid to homocysteine and for then supplying this unit to the lipid metabolism. Vitamin B6 participates in a metabolic branch for the re-methylation, and indirectly acts on the lipid metabolism.

The dosages disclosed in this application refer to amounts by weight of the agents folic acid, vitamin B6, and vitamin B12, respectively, so that for salts or derivatives, the respective recalculation has to be effected. The same holds analogously for the agent percentages disclosed in the present specification. Alternatively, the ratios can be related to the molar amounts, so that, assuming 1 mol of the respective derivative or salt to contain 1 mol folic acid, vitamin B6 or vitamin B12, respectively, the molar ratios for folic acid, vitamin B6, vitamin B12 and their derivatives and/or salts may be expressed in a unified manner.

“Folic acid” according to this invention designates N-pteroylglutamic acid of formula I,

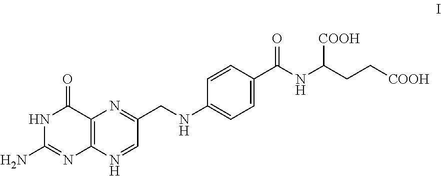 [SMILES: Nc1nc2ncc(CNc3ccc(C(=O)NC(CCC(=O)O)C(=O)O)cc3)nc2c(=O)[nH]1]

including the optical isomers matching this formula as mixtures, e.g. as a racemate, as well as in pure form, e.g. R- or S-enantiomers. Particularly preferred is N-pteroyl-L-glutamic acid of formula Ia:

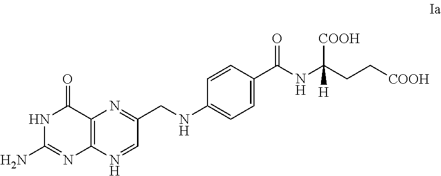 [SMILES: Nc1nc2ncc(CNc3ccc(C(=O)N[C@@H](CCC(=O)O)C(=O)O)cc3)nc2c(=O)[nH]1]

To the folic acid derivatives belong, in particular, folic acid metabolites as well as amides and esters of folic acid as well as of the metabolites. Particularly preferred are amides and esters which are hydrolyzable under physiologic conditions, like amides with C₁-C₁₀-alkyl amines or esters with C₁-C₁₀-alcohols. A particular form of the amides are N-pteroyl-polyglutamic acids.

To the folic acid metabolites belong, in particular, H₄-folic acids of formula Ib

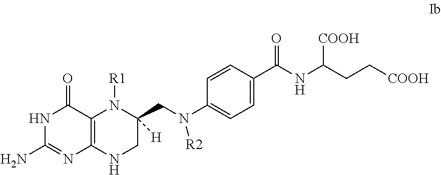

wherein R1 is hydrogen, methyl, —HC═O (formyl) or —HC═NH (formimino) and R2 is hydrogen or —HC═O (formyl), or R1 and R2 taken together form a methylene or methenyl bridge. The optical isomers matching the formula are included, wherein again the L-glutamic acid derivatives are preferred. In particular, tetrahydrofolic acid, 5-methyltetrahydrofolic acid, 5,10-methylenetetrahydrofolic acid, 5-formyl-tetrahydrofolic acid, 10-formyltetrahydrofolic acid, 5-formiminotetrahydrofolic acid, and 5,10-methenyltetrahydrofolic acid are to be mentioned.

To the physiologically acceptable salts of folic acid or folic acid derivatives belong acidic and basic addition salts as well as the respective mixed forms.

To the acidic addition salts belong salts of folic acid derivatives with inorganic acids, such as hydrochloric acid, sulfuric acid, nitric acid, or phosphoric acid, or organic acids, in particular carboxylic acids, e.g. acetic acid, tartaric acid, lactic acid, citric acid, malic acid, mandelic acid, ascorbic acid, maleic acid, fumaric acid, gluconic acid, or sulfonic acids, e. g. methanesulfonic acid, benzenesulfonic acid and toluenesulfonic acid.

To the basic addition salts belong salts of folic acid or folic acid derivatives with inorganic bases, for example metal hydroxides or carbonates of alkaline, alkaline earth or transition metals, or with organic bases, for example ammonia or basic amino acids, such as arginine and lysine; amines, e.g. methylamine, dimethylamine, trimethylamine, triethylamine, ethylamine, diethylamine, ethylenediamine, ethanolamine, diethanolamine, 1-amino-2-propanol, 3-amino-1-propanol or hexamethylenetetramine; saturated cyclic amines with 4 to 6 ring carbon atom, such as piperidine, piperazine, pyrrolidine, and morpholine; and further organic bases, for example

N-methylglucamine, creatine and tromethamine, as well as quaternary ammonium compounds, such as tetramethylammonium and the like.

Salts with inorganic bases are preferred, e.g. Na-, K-, Mg-, Ca-, Zn-, Cr-, and Fe-folate.

“Vitamin B6” in the context of this application designates 4,5-bis(hydroxymethyl)-2-methyl-3-pyridinol of formula II

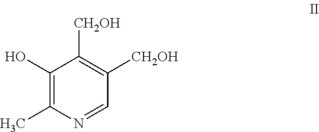

also termed pyridoxine (INN).

To the vitamin B6-derivatives belong mainly pyridoxals and pyridoxamines as well as Esters of the pyridoxines, pyridoxals and pyridoxamines. Preferred are again esters which are hydrolyzable under physiological conditions. In particular, in this conjunction the pyridoxines, pyridoxals and pyridoxamines of formula IIa should be named:

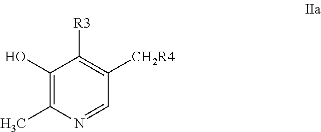

wherein R3 indicates CH₂OH, CHO or CH₂NH₂ and R4 indicates OH or OPO₃H₂.

To the physiologically acceptable salts of vitamin B6 or vitamin B6-derivatives belong in particular acidic addition salts, e.g. with the above mentioned inorganic or organic acids. In particular, the hydrochloride, mainly pyridoxin-HCl, should be mentioned.

“Vitamin B12” is also known as cyanocobalamine or cobalamine, formula III:

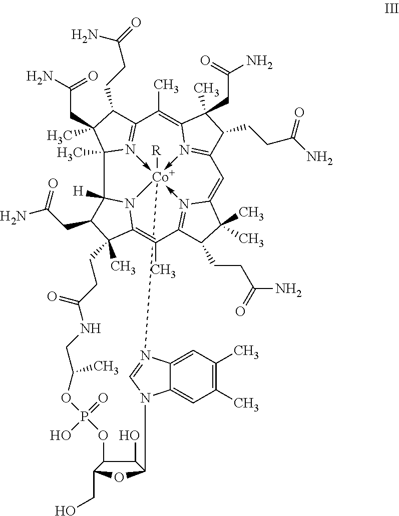

wherein R represents a cyano group. To the vitamin-B12-derivatives belong in particular cobalamines in which the cyano group (“R”) of the cyanocobalamine is replaced by other coordination partners of the cobalt atom. In this context, mainly aquocobalamine (“Vitamin B12a”), hydroxocobalamine (“Vitamin B12b”), nitroso-cobalamine, methylcobalamine (“Vitamin MeB12”) and adenosylcobalamine (Coenzym B12) should be mentioned. To the physiologically acceptable salts of vitamin B12 or vitamin B12-derivatives, respectively, belong in particular acidic addition salts e.g. with the above mentioned inorganic and organic acids. In particular, the acetate of hydroxocobalamine should be mentioned.

Folic acids, B6- and B12-vitamins are, as such, well known to the skilled person and may be commercially obtained or provided in a different manner.

Along with the folic acid, vitamin B6- and vitamin-B12-components, the composition according to some embodiments may contain further ingredients and agents. Such agents may in particular be ones the effect of which is similar to that of folic acid, vitamin B6 or vitamin B12, respectively, or enhances same, and in particular corresponds to the present purposes. Such agents are mainly choline (daily dose at least 10 mg, e.g. 90-500 mg), grape seed extract (daily dose at least 10 mg, e.g. 40-500 mg) and/or artichoke extract (daily dose at least 10 mg, e.g. 40-500 mg). Also carotenoids, in particular xanthophylls, further in particular astaxanthine may additionally be administered. Moreover, further vitamins such as vitamin A, vitamin C, vitamin D and/or vitamin E may be added. In particular, vitamin B2 vitamin B3 may be present in the composition. Suitable daily doses are, depending on the agent, at least in the range of 0.1-10 mg. In addition, betaine and/or statins, in particular monacolin K and/or omega-3-fatty acids may be present.

### DETAILED DESCRIPTION OF EXEMPLARY EMBODIMENTS

The treatment with the composition of some embodiments is particularly called for when the HDL-value is lowered and/or the LDL-Wert and/or the LDL/HDL-ratio is elevated; but also when cholesterol alone, triglycerides alone or both cholesterol and triglycerides are elevated. In the context of this application, an elevation of the LDL/HDL-ratio is considered to be an LDL/HDL-ratio of more than 2.5, in particular more than 3.0, more in particular of more than 3.5 or even of more than 4.0. The compositions of some embodiment usually become ever more important with increasing age of an adult person.

In a 3-month study with ca. 350 patients, upon administration of a daily dose of 1 mg folic acid, 1 mg vitamin B12 and 50 mg vitamin B6 the proportion of patients having an LDL-value above the reference value lowered from more than 36% to less than 22%. Moreover, on average the cholesterol value and the triglyceride value lowered, while the HDL-value rose. The changes were the more pronounced, the farther away the original values were from the respective reference value.

According to embodiments, the individual to be treated is administered a sufficient amount of the inventive agent composition of a folic acid component, a vitamin B12-component and, if desired, a vitamin B6-component, as a rule formulated according to the practice of the pharmaceutical or nutritional-technological field, is orally administered. According to embodiments, the amount is sufficient in particular if it results in a significant lowering of the blood lipid level, advantageously to within the normal range. Particularly advantageous is a lowering of the LDL/HDL-ratio to below 4.0, preferably to below 3.5, more preferably to below 3.0, still more preferably to below 2.5.

As a general rule, the treatment is effected by a once-daily or plural-daily administration, or an intermittent administration (i.e. with no administration for one or plural days up to a week) of a sufficient dose, if desired together or alternatingly with other agents or agent-containing formulations, so that an individual to be treated, assumed to have the average adult body weight of about 75 kg, as a general rule is administered minimum average daily doses of about 0.2 mg, preferably about 0.5 mg and more preferably about 0.6 mg folic acid; of about 4 mg, preferably about 10 mg and more preferably about 20 mg vitamin B6 (if present); and of about 8 μg, preferably about 0.1 mg and more preferably about 0.2 mg vitamin B12. On the other hand, the administered maximum average daily dose as a general rule is about 10 mg, preferably about 1.1 mg and more preferably about 0.9 mg folic acid, about 100 mg, preferably about 75 mg and more preferably about 50 mg vitamin B6, and about 1.2 mg, preferably about 0.8 mg and more preferably about 0.5 mg vitamin B12.

As a general rule, the treatment is continued for a suitable period in the range of several weeks or months. Preferred is a normalization of the lipid level within a treatment period of about 1 month to about 6 months. In necessary, the treatment is continued even after the normalization of the lipid level.

To the pharmaceutical compositions belong in particular nutritional supplements and functional or dietetic food. According to embodiments, functional or dietetic food has an agent-related function in addition to its main, nutritional function. They are therefore termed functional or dietetic food or nutrition. Nutritional supplements serve the augmentation of the daily nutritional intake with an agent combination according to some embodiments, wherein the nutritional function of the nutritional supplement as such, is less relevant.

According to some embodiments the present invention relates to formulations containing

i) at least one agent selected from the folic acid group (i.e. folic acid, physiologically acceptable derivatives thereof and salts thereof),
ii) at least one agent selected from the vitamin B12-group (i.e. vitamin B12, physiologically acceptable derivatives thereof and salts thereof), and optionally
iii) at least one agent selected from the vitamin B6-group (i.e. vitamin B6, physiologically acceptable derivatives thereof and salts thereof), as well as, if desired, at least one further agent and a formulating basis, in the amounts stated above.

In other words, the agent combination according to some embodiments comprises, as the agent component i) at least one selected from the group consisting of folic acid, a physiologically acceptable derivative thereof and a salt thereof. Mixtures thereof are also envisioned. According to a preferred embodiment, the agent component i) comprises at least 90% by weight folic acid.

Moreover, the agent combination according to some embodiments comprises, as the agent component ii) at least one selected from the group consisting of vitamin B12, a physiologically acceptable derivative thereof and a salt thereof. Mixtures thereof are also envisioned. According to a preferred embodiment, the agent component ii) comprises at least 90% by weight cobalamin.

Moreover, the agent combination according to some embodiments comprises, as the agent component iii) at least one selected from the group consisting of vitamin B6, a physiologically acceptable derivative thereof and a salt thereof. Mixtures thereof are also envisioned. According to a preferred embodiment, the agent component comprises at least 90% by weight pyridoxine-HCl.

The proportion of the agent combination in the formulation is, in embodiments, larger than that found in natural sources, in particular in foods. In this regard, the composition according to some embodiments is enriched in respect of agent combination. The proportion of the agent combination of i), ii) and iii) in the formulation preferably is at least about 0.01% by weight, more preferably is at least about 0.05% by weight, and still more preferably is at least about 0.1% by weight. In the case of a pharmaceutical composition, the proportion as a general rule is about 1 to 60% by weight, preferably about 5 to 35% by weight, and still more preferably about 10 to 30% by weight; in the case of nutritional supplements and more particularly in the case of foods the proportion may be accordingly less, if the formulation is administered in larger amounts. According to embodiments, the formulations contain the daily doses as stated above.

Any values given in % by weight refer to the total weight of the formulation. According to embodiments, the formulation base of pharmaceutical formulations comprises physiologically acceptable adjuvants or excipients. Physiologically acceptable are those adjuvants and excipients known to be useable in the field of pharmacy, nutritional technology and neighboring fields, in particular those listed in medical handbooks (e.g. DAB, Ph. Eur., BP, NF), and also other adjuvants and excipients the properties of which do not interfere with their physiological use. Adjuvants and excipients may themselves have a nutritional value and may therefore be generally used as nutritional components. Also, nutritional components, in particular essential nutritional components, may belong to this group.

Useful adjuvants or excipients may comprise: surfactants; emulsifying or suspending agents; preserving agents; antioxidants; anti-irritants; chelate formers; dragee formers; emulsion stabilisers; film formers; gelling agents; olfactoric masking agents; taste modifiers; resins; hydrocolloids; solvents; solubilizing agents; neutralising agents; permeation enhancers; pigments; quaternary ammonium compounds; re-fatting and overfatting agents; ointment, cream, or oil base components; silicone derivatives; spreading agents; stabilisers; sterilisers; tablet adjuvants such as binders, fillers, lubricants, disintegrants, or coatings; bubbling agents; drying agents; opacifiers; thickeners; waxes; softeners; white oils. Such additives are known to the skilled person, for example as set out in Fiedler, H. P., “Lexikon der Hilfsstoffe für Pharmazie, Kosmetik and angrenzende Gebiete,” 5. Ed., ECV⋅Editio Cantor Verlag, Aulendorf 2002.

Nutritional components usually contain one or more amino acids, carbohydrates or fats and are suitable for human nourishment. They comprise single components, often herbal, but also of animal origin products, in particular sugars, if suitable in the form of sirups, fruit compositions such as fruit juices, nectar, fruit pulps, purees, or dreid fruits, for example apple juice, grapefruit juice, orange juice, apple puree, tomato sauce, tomato juice, tomato puree; cereal products such as wheat flour, rye flour, oatmeal, corn flour, barley flour, spelt flour, corn sirup, as well as starches of the cereals mentioned; and milk products such as milk protein, whey, yoghurt, lecithin and milk sugar.

To the essential nutrients belong in particular vitamins, pro-vitamins, trace elements, amino acids and fatty acids. As essential amino acids may be named isoleucine, leucine, lysine, methionine, phenylalanine, threonine, tryptophan and valine. Also, semi-essential amino acids belong in this group, which have to be supplied in certain growth periods or states of shortage, such as arginine, histidine, cysteine and tyrosine. As trace elements may be mentioned: essential trace elements, the necessity of which for humans is proven and the deficiency of which leads to manifestation of clinical symptoms: iron, copper, zinc, chromium, selenium, calcium, magnesium, potassium, lithium, cobalt, molybdenum, iodine, silicon, fluorine. Also, elements the function of which is not yet sufficiently ascertained for humans: tin, nickel, vanadium, manganese. As fatty acids essential for humans may be mentioned: linoleic acid and the omega-3-fatty acids linolenic acid, eicosapentaenic acid (EPA) and docosahexaenic acid (DHA). A comprehensive listing of vitamins may be found in “Referenzwerte für die Nährstoffzufuhr”, 2. Ed., Umschau Braus Verlag, Frankfurt am Main, 2015, edited by the Deutsche Gesellschaft für Ernährung. The sum of actual agents and formulating base in general is 100% by weight.

Examples of suitable formulations for nutritional supplements include capsules, tablets, pills, powder baglets, liquid containing ampoules, and bottles with drop inserts, as well as the following medicinal forms:

Examples of suitable pharmaceutical formulations are solid medicinal forms such as powder, granules, tablets, in particular film tablets, pastilles, sachets, cachets, dragees, capsules such as hard or soft gelatin capsules; semi-solid medicinal forms such as ointments, creams, hydrogels or pastes; and liquid medicinal forms such as solutions, emulsions, in particular oil-in-water emulsions, suspensions, for example lotions. Moreover, implanted dispensing devices may be used for the administration of the agents. Furthermore, liposomes or microspheres may be employed.

Nutritional-technological formulations are preferably provided in the shape of infant nutrition, breakfast compositions, such as muesli or bars, sport drinks, entire meals, in particular in the context of fully balanced diets, dietetic formulations such as dietary drinks, dietary meals and dietary bars. The formulations are preferably administered orally.

In the manufacture of the compositions, according to embodiments the agents are typically mixed or diluted with an excipient. Excipients may comprise solid, semi-solid or liquid materials acting as vehicle, carrier, or medium for the agent. The admixing of further excipients or adjuvants can be performed, if required, according to methods known in the art. Shaping steps, if required in combination with mixing procedures, may also be performed, e.g. granulation, compartmentalization and the like.

In particular, the agent components may be formulated together. Alternatively, they can be process separately and subsequently combined in a compartmentalized shape, e.g. a multi-layer medicinal form. Thereby, potential agent incompatibilities and differing agent properties such as bioavailability, stability, solubility, and the like, may be taken account of.

The above disclosure referred exemplarily to a lowering of the LDL/HDL-ratio. However, the regulation of the lipid metabolism may also show up in terms of an elevation of the HDL-value, or a lowering of the total cholesterol value, the LDL-value, the triglyceride value, or both the cholesterol value and the triglyceride value, depending on the starting point: In general, the regulation mainly consists of a normalization of conspicuous values (according to their deviating from normal, or reference values), whereas inconspicuous values are less affected. Alongside with these changes, in addition or alternatively, the creatinine value and the blood glucose level may be advantageously affected.

While the disclosure has been described with respect to certain exemplary embodiments thereof, it is evident that many alternatives, modifications and variations will be apparent to those skilled in the art. Accordingly, the exemplary embodiments of the disclosure set forth herein are intended to be illustrative and not limiting in any way. Various changes may be made without departing from the spirit and scope of the present disclosure as defined in the following claims.

1. A method of treating a lipid disorder, comprising administering to a subject in need thereof a pharmaceutical composition comprising:
folic acid, a physiologically acceptable derivative thereof, or a salt thereof; and
vitamin B12, a physiologically acceptable derivative thereof, or a salt thereof,
wherein a daily dose of the composition comprises 0.2 to 10 mg folic acid, the physiologically acceptable derivative thereof, or the salt thereof; and at least 8 μg vitamin B12, the physiologically acceptable derivative thereof, or the salt thereof.

2. The method of claim 1, wherein the pharmaceutical composition further comprises vitamin B6, wherein a daily dose comprises at least 4 mg vitamin B6.

3. The method of claim 2, wherein the daily dose of vitamin B6 is at most 500 mg.

4. The method of claim 3, wherein the daily dose of vitamin B6 is more than 10 mg, and at most 100 mg.

5. The method of claim 3, wherein the daily dose of vitamin B6 is at most 25 mg.

6. The method of claim 1, wherein the daily dose of folic acid, the physiologically acceptable derivative thereof, or the salt thereof, is at least 0.5 mg, and at most 1.2 mg.

7. The method of claim 6, wherein the daily dose of folic acid, the physiologically acceptable derivative thereof, or the salt thereof, is at most 0.8 mg.

8. The method of claim 1, wherein the daily dose of vitamin B12, the physiologically acceptable derivative thereof, or the salt thereof, is at least 0.1 mg, and at most 4 mg.

9. The method of claim 8, wherein the daily dose of vitamin B12 the physiologically acceptable derivative thereof, or the salt thereof, is at most 0.8 mg.

10. The method of claim 1, wherein a ratio of the daily dose of folic acid, the physiologically acceptable derivative thereof, or the salt thereof, to the daily dose of vitamin B12, the physiologically acceptable derivative thereof, or the salt thereof, is at least 0.2.

11. The method of claim 10, wherein the ratio of the daily dose of folic acid, the physiologically acceptable derivative thereof, or the salt thereof, to the daily dose of vitamin B12, the physiologically acceptable derivative thereof, or the salt thereof, is at least 0.8.

12. The method of claim 1, wherein the pharmaceutical composition further comprises choline, wherein the daily dose of choline administered with the composition is at least 10 mg.

13. The method of claim 12, wherein the daily dose of choline administered with the composition is 90 to 700 mg.

14. The method of claim 1, wherein the pharmaceutical composition further comprises grape seed extract, wherein the daily dose of grape seed extract administered with the composition is at least 10 mg.

15. The method of claim 14, wherein the daily dose of grape seed extract administered with the composition is 40 to 700 mg.

16. The method of claim 1, wherein the pharmaceutical composition further comprises artichoke extract, wherein the daily dose of artichoke extract administered with the composition is at least 10 mg.

17. The method of claim 16, wherein the daily dose of artichoke extract administered with the composition is 40 to 700 mg.

18. The method of claim 1, wherein the pharmaceutical composition further comprises at least 0.5 mg riboflavin.

19. The method of claim 1, wherein the pharmaceutical composition further comprises at least 5 mg niacin.

20. The method of claim 1, wherein the pharmaceutical composition further comprises a carotenoid.

21. The method of claim 20, wherein the carotenoid is a xanthophyll.

22. The method of claim 21, wherein the xanthophyll is astaxanthin and its daily dose is at least 0.1 mg.

23. The method of claim 1, wherein the pharmaceutical composition further comprises a statin.

24. The method of claim 23, wherein the statin is a monacolin and its daily dose is at least 1 mg.

25. The method of claim 24, wherein the statin is monacolin K, compactin, or both.

26. The method of claim 1, wherein the pharmaceutical composition further comprises betaine, the daily dose of which is at least 5 mg.

27. The method of claim 1, wherein the pharmaceutical composition further comprises an omega-3-fatty acid, the daily dose of which is at least 20 mg in total.

28. The method of claim 1, wherein the pharmaceutical composition is administered to a person without hyperhomocysteinemia.

29. The method of claim 1, wherein the pharmaceutical composition is administered to a person with an elevated blood glucose level, with an elevated creatinine level, or both.

30. The method of claim 1, wherein the lipid disorder is at least one selected from the group consisting of dislipidemia, dislipoproteinemia, and hypertriglyceridemia.
